(19) United States
(12) Patent Application Publication
(10) Pub. No.: US 2002/0165270 A1
(43) Pub. Date: Nov. 7, 2002

(54) Use of a pharmaceutical composition for treating and/or preventing ischemia

(76) Inventors: Jose Remacle, Malonne (BE); Carine Michiels, Spy (BE)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 10/131,921

(22) Filed: Apr. 23, 2002

Related U.S. Application Data
(63) Continuation of application No. 09/423,967, filed on Mar. 20, 2000, now abandoned.
Application No. 09/423,967 is a 371 of international application No. PCT/BE98/00067, filed on May 12, 1998.

(30) Foreign Application Priority Data
May 13, 1997 (BE) 9700415

(51) Int. Cl. A61K 31/365; A61K 35/78

(52) U.S. Cl. 514/468; 424/752

(57) ABSTRACT

The present invention relates to a method for therapeutically and/or prophylactically treating ischemia and/or the pathologies associated with ischemia or an energy deficit in a patient comprising administering to the patient a pharmaceutical composition comprising bilobalide and a pharmaceutically acceptable excipient where the composition has a therapeutic and/or prophylactic effect on ischemia and/or the pathologies associated with ischemia or an energy deficit.

### OBJECT OF THE INVENTION

The present invention relates to the therapeutic and/or prophylactic application of a pharmaceutical composition in the treatment and/or prevention of ischemia and of pathologies which are associated with ischemia.

### STATE OF THE ART AND TECHNOLOGICAL BACKGROUND

Various active compounds, most of which are present in plant extracts, have been proposed for treating certain varicoses diseases. Such compounds are described, in particular, in the following documents: French Patent Application FR-2692145, French Patent Application FR-2668705, European Patent EP-0541874-B1, International Patent Application W093/20046, International Patent Application W093/20045, European Patent EP-0566445-B1, European Patent Application EP-0210781-A1 and European Patent Application EP-0112770-A1.

Similarly, active compounds isolated from plant extracts have been used for treating the consequences of ischemia or pathologies associated with ischemia. This is the case with flavonoids, which are known to be antioxidants and which are able to limit the damage which is caused by the free radicals which are produced during reperfusion. Thus, when reperfusion takes place after a period of ischemia, a very considerable production of oxygen-derived free radicals is observed, which production will cause damage to the various constituents of the cell, which has been weakened by the period of ischemia, in particular by the lack of energy (ATP). This increased production of free radicals was clearly demonstrated by McCord J. M. (1985, N. Engl. J. Med., 312: 159-163). It is due to xanthine oxidase being activated during the period of ischemia and to the very high activity of this enzyme during reperfusion. Antioxidant molecules such as the flavonoids therefore have a beneficial effect on the ischemia reperfusion process since they protect the tissue against this excess of free radicals (Reddy D. S. et al., 1995, Drugs of today, 31: 335-349).

However, many plant extracts consist of mixtures of large numbers of complex macromolecules, and the presence of some of these macromolecules of structures of the flavonoid type which are known for their antioxidant properties has overshadowed the possibility of these extracts or molecules having an anti-ischemic activity which is peculiar to them, that is which is independent of the process of reoxygenation.

The activity of the flavonoids on ischemia and in decreasing the impact of myocardial infarction is due to this activity in controlling the active oxygen derivatives (Reddy et al., 1995, Drugs of today, 31: 335-349; DE-3623255, OXO Chimie GMBH, 1988). Troxerutin has, in particular, been used as an antioxidant molecule which protects the heart during the process of ischemia reperfusion (Blasug I. E. et al., 1987, Biomed. Biochim. Acta, vol. 46: 5539-544; XP 002052079 et Olszenski A. J., 1991, Atheroschlerosis, 88: 97-98).

Similarly, diosmin, which blocks the formation of free radicals by xanthine oxidase (which take [sic] place during the reperfursion after the ischemia), has been used in this sense as being able to protect during ischemia (Bouskela E. et al., 1997, 45: 33-37; Int. J. Microcirc. Clin. Exp., 1995, 15: 293-300; Debbarre B. et al., 1995, Int. J. Microcirc. Clin. Exp., 15 suppl. I: 27-33).

Rutosides have also been successfully tested for their anti-ischemia activity. Thus, they reduce the magnitude of the impact in animals which have been subjected to an arterial occlusion (Zalewski A. et al., Am. J. Cardiol., 1985, 56: 974-977), and also provide relief for patients suffering from ischemia of the lower limbs (Milliken J. C., Vasa, 1974, 3: 203-206).

Nafthoquinone, which is known for its activity in preventing platelet aggregation, can also be used to inhibit the formation of the thrombus during thromboses (EP-A-0631777).

The demonstration of this activity does not constitute an anti-ischemic activity as such but, instead, an action on one of the causes of thrombosis, namely aggregation of the platelets.

### OBJECTS OF THE INVENTION

The present invention is directed towards providing a novel process for the therapy and/or prophylaxis of ischemia and/or pathologies which are associated with ischemia.

A particular object of the present invention is directed towards achieving a process which uses therapeutic agents which are not toxic or not particularly toxic, which exhibit few or no side-effects and whose synthesis or extraction from live products, in particular from plants, is simple and inexpensive.

### CHARACTERISTIC ELEMENTS OF THE INVENTION

The present invention relates to the use of a pharmaceutical composition, which comprises a pharmaceutical excipient and a sufficient quantity of an active compound which is selected from the group consisting of citroflavonoids, garlic-rutoside, troxerutin, coumarine, diosmin, o-(hydroxyethyl) rutosides, melilot and rutoside extracts, tribenoside, hesperidin methyl chalcone, horse-chestnut extract, naftazone, esculoside, aescin, procyanidine oligomers, ruscus and hesperidin methyl chalcone extracts, ruscosides, common holly and blackcurrant extracts and blueberry anthocyanin extracts and the active principles which are isolated from these compounds, and/or a mixture thereof, for preparing a medicament which is intended for treating and/or preventing ischemia and/or pathologies which are associated with ischemia.

The abovementioned active compounds are products which are widely isolated from plants and/or from plant extracts and which are marketed by various pharmaceutical firms under various brand names. These different active compounds, which are identified by their brand name and the companies which market them, are listed in Table 1 below.

TABLE 1
Active compounds of the composition according
to the invention

| Active principle | Brand name ® | Company |
|---|---|---|
| Citroflavonoids | Agruton C | Sanofi-Winthrop |
| Citroflavonoids | Daflon | Eutherapie- |
| | | Servier |
| Garlic-rutoside | Ex-Ail | Solvay |
| Tribenoside | Glyvenol | Ciba-Geigy |
| Hesperidin methyl | Hemocoavit | Wynlit/Bio- |
| chalcone | | Therabel |
| Horse-chestnut | Intrait de | Pharmethic |
| | Marron d′Inde | |
| Esculoside | Mictasol-P | Medgenix |
| Aescin | Reparil | Madaus |
| Procyanidine | Endothelon | Sanofi |
| oligomers | | |
| Ruscus and | Cyclo 3 | Fabre |
| hesperidin methyl | | |
| chalcone extracts | | |
| Ruscosides | Cirkan | Sinbio-Fabre |
| Common holly and | Veinobiase | Laboratoire |
| blackcurrant | | Fournier |
| extracts | | Schwartz-Pharma |
| Blueberry | Difrarel | Labo Leurquin |
| anthocyanin | | Mediolanum |
| extracts | | |

Table 2 lists the active compounds of the composition according to the invention which have already been shown to have a protective capacity in the ischemia reperfusion process.

TABLE 2
Active compounds of the composition according
to the invention which have already been shown to have
a protective capacity in the ischemia reperfusion
process

| Active principle | Brand name ® | Company |
|---|---|---|
| Troxerutin | Veinamitol | Vitalpharma |
| Coumarin troxerutin | Venalot-Depot | Boots |
| Diosmin | Ven Dretex [sic] | Therabel |
| o-(hydroxyethyl) | Venox | Eumedis/Therabel |
| rutosides | | |
| Diosmin | Daflon | Servier |
| Melilot and | Esberiven | Knoll |
| rutoside extracts | | |
| Troxerutin | Reoflux | Negma |
| Diosmin | Dioveinor [sic] | Imnothera |

These active compounds, and their posology and their preferred forms of administration, are also described in the documents “Répertoire commenté des médicaments” [annotated list of medicines] (Centre Belge d'Information Pharmaco-Thérapeutique, [Belgium Centre for Pharmaco Therapeutic Information], Brussels, 1994) and Vidal 1997 (ed. Du Vidal, 33 Av. de Wagram, Paris, France). “Active principle which is isolated from an active compound of the invention” is understood as being the active part which has a therapeutic and/or prophylactic effect with regard to its biochemical target, as described below, and which is likely to have properties, in the therapeutic and/or prophylactic sphere, which are comparable and/or superior to those of the active compound described below.

The active principles and compounds of the invention are also characterized by having a “protective effect” on the protein complexes of the internal mitochondrial membrane, that is to say that the active principles and compounds of the invention are able to increase the RCR (Respiratory Control Ratio), which represents the mitochondrial respiratory control, of a patient. The RCR represents the ratio between the consumption of oxygen in the presence of endogenous substrates (glutamate/malate) and the consumption after phosphorylation of the ADP into ATP, as will be illustrated below.

The products of the invention use this mechanism of action to protect the patient from the ischemia or the consequences of the ischemia. The active compounds and principles of the invention are therefore characterized at one and the same time by a prophylactic and/or therapeutic effect.

Preferably, the products of the invention have a protective effect on complex I or complex III of the electron transport chain in the mitochondria and/or on the adenine translocase protein complex, as illustrated below.

The preferred active compounds of the invention are hesperidin methyl chalcone, aescin, procyanidine oligomers and blueberry anthocyanin extracts, which active compounds are characterized by properties which are particularly advantageous and unexpected in the treatment of ischemia and pathologies which are associated with ischemia, with an energy deficit and with deficiencies linked to ageing.

“Sufficient quantity of an active principle or compound” is understood as being a quantity of this active principle or compound which is sufficient for treating, relieving, dissipating or alleviating the symptoms or the dysfunction of the human or animal body which- are associated with the abovementioned disorders and/or for preventing or decreasing the possibility of being affected by them. Consequently, the implementation of the abovementioned therapeutic treatment relates to a prophylactic treatment or a curative treatment of the said disorders. The percentage of this active compound can vary over a very wide range, which range is only limited by the tolerance, and the level of acceptance, of the compound by the patient. These limits are determined, in particular, by the frequency of administration.

Preferably, the doses are the doses of these products which are generally used in treating varicose disorders, as described in the document “Répertoire commenté des médicaments, Centre Belge d'Information Pharmacothérapeutique, Bruxelles (1994)” and in the document “Médicaments utilisés pour le traitement symptomatique des affections veineuses périphériques, [Medicines used for the symptomatic treatment of peripheral venous ailments], Annexe des folia pharmacotherapeutica, Ministère Belge de la Santé Publique et de l'Environnement, Commission de Transparence (juin 1994) [Annex to the Folia pharmacotherapeutica, Belgian Ministry of Public Health and the Environment, Transparency Commission (June 1994))”. By way of example, the product aescin, which is sold under the brand name Reparil®, is presented in the form of sugar-coated tablets having an active compound dose of 20 mg in a pharmaceutical composition of 100 mg.

The pharmaceutical excipient employed varies depending on the chosen mode of administration (intravenous, intramuscular, oral, etc.) and can have different forms such as coated or uncoated tablets, pills, capsules, solutions, syrups, etc.

The pharmaceutical compositions will be prepared using methods which are generally used by galenists and pharmacists and which can include any type of solid, liquid and/or gaseous (including water) pharmaceutical excipient which is not toxic or not particularly toxic.

The pharmaceutical composition according to the invention can also include an adjuvant or another pharmaceutical compound which is known, by the skilled person, for its therapeutic and/or prophylactic effects on the abovementioned disorders or for its properties which are likely to decrease the side-effects which are associated with the active compound which is present in the pharmaceutical composition of the invention.

Furthermore, the bioavailability of the active compounds of the invention within the organism, in particular as a result of their possible resorption and their passage into the target tissue to be protected, can be improved by techniques of packaging and coating and/or drug-targeting which are well known to the skilled person.

“(Partial or total) ischemia or pathologies associated with ischemia” are understood as being vascular disorders which are selected from the group consisting of myocardial infarction, cerebral ischemia, chronic venous insufficiency, atheriopathies, that is lesions which are due to the atheroschlerosis which affects the arteries of the patients, Raynaud's phenomenon linked to vasospasms, leading to vasoconstriction of the arteries, ulcers, change in capillary permeability, capillary fragility, wound-healing, changes to the skin, retinal defects of ischemic origin, loss of auditory acuity of ischemic origin, disorders associated with time spent at high altitude, angina pectoris engendered by short periods of coronary obstruction, pulmonary hypertension, hepatic ischemia, Parkinson's disease, myopathies and syndromes associated with vascular problems such as diabetes, where hypertension and a change in the blood flow appear in the lower limbs. These ischemia-linked disorders and pathologies are well known to clinicians and doctors, who can adjust the use of the pharmaceutical composition for treating and/or preventing the symptoms and dysfunction of a human or animal body which are associated with the abovementioned disorders and/or for preventing or decreasing the possibility of being affected by them.

Another aspect of the present invention relates to the use of the pharmaceutical composition according to the invention when there is a decrease, which is associated with ageing, in the production of energy by the various different cells. This applies in the case of intellectual failings in an elderly subject, of the vertigo syndrome and of the fall in perspective accommodation which is due to an alteration in metabolic regulation.

A final aspect of the present invention relates to the use of the pharmaceutical composition according to the invention in the transplant field.

The said pharmaceutical composition can be used directly on the patient or be intended for an ex-vivo treatment of the patient in which an organ, a tissue and/or a physiological liquid, such as the blood or serum, which is derived from the patient himself or from another human or animal patient is treated by the said pharmaceutical composition according to the invention being added directly to the organ, to the tissue or to the physiological liquid prior to its being administered to the patient. This application relates, in particular, to the field of heart transplants.

The present invention also relates to a process for therapeutically and/or prophylactically treating the ischemia and/or the pathologies associated with the ischemia or with an energy deficit as well as the deficiencies linked to ageing, such as the intellectual failings of the elderly subject, the vertigo syndrome or the fall in perspective accommodation due to an alteration in metabolic regulation, of a patient, in which process the pharmaceutical composition according to the invention is administered to said patient so as to treat, relieve, dissipate or alleviate the symptoms or dysfunction of the human or animal body which are associated with the abovementioned disorders and/or to prevent or decrease the possibility of being affected by them. This administration is performed using the methods which are well known to the skilled person, in particular those which are used for treating varicose disorders.

These prophylactic and/or therapeutic effects are described, in particular, with reference to the figures, in the examples which are appended hereto and which are given by way of illustrating the subjectmatter of the invention in a non-limiting manner.

### BRIEF DESCRIPTION OF THE FIGURES

FIG. 1 diagrammatically depicts the cascade by which the endothelial cell is activated by hypoxia, and its consequences for the adherence and activation of the neutrophils.

FIG. 2 diagrammatically depicts the cascade by which the endothelial cells are activated by hypoxia and its consequences for the blood-vessel wall.

FIG. 3 depicts the measurement of mitochondrial respiratory activity as different active compounds are added.

FIG. 4 diagrammatically depicts the electron transport chain in the internal mitochondrial membrane.

### EXAMPLES

As a result of their location at the blood-tissue interface, the endothelial cells (EC) are responsible for maintaining vascular homeostasis. They thus fulfil a whole series of functions and interact constantly with the circulating leukocytes and the smooth muscle cells (SMC) of the tunica media. Any disruption in their metabolism can therefore bring about changes in the functioning of the underlying tissues.

Because they are located at the blood-tissue interface, the ECs are-the first to suffer from any alteration in blood flow, in particular a decrease in this blood flow during stases, which lead to an impoverishment in the supply of oxygen and nutrients to the tissues (Hinshaw et al., 1988; Tsao et al., 1990).

Hypoxia, which can, in particular, result from such a stasis, has a marked activating effect on the ECs, which release inflammation mediators which are able to activate neutrophils, and induce them to infiltrate, as well as growth factors for the SMCs. This cascade of events finally leads to structural and functional alterations of the venous wall.

### Effect of Hypoxia on Endothelial Functions

In order to study the alterations in the metabolism of the ECs when the supply of oxygen is reduced, ECs which have been isolated from the human umbilical vein are incubated in vitro under hypoxia. Under these experimental conditions, none of the ECs is seen to die during the first two hours of incubation. However, substantial changes are seen in their metabolism; the ECs are strongly activated by the hypoxia in a manner which is similar to the activation which is initiated by thrombin or histamine.

The first sign of this activation is an increase in the concentration of calcium in the cytosol [Arnould et al., 1992]. This increase is linked to a decrease in the concentration of ATP. Calcium is an important second messenger in all cells. In the ECs, it is, in particular, able to activate phospholipase A₂, which is the first enzyme in a metabolic pathway leading to the synthesis of inflammation mediators. This activation leads to an increased synthesis of prostaglandins [Michiels et al., 1993]. It also induces the synthesis of platelet activating factors (PAF), which is a very powerful inflammation mediator [Arnould et al., 1993].

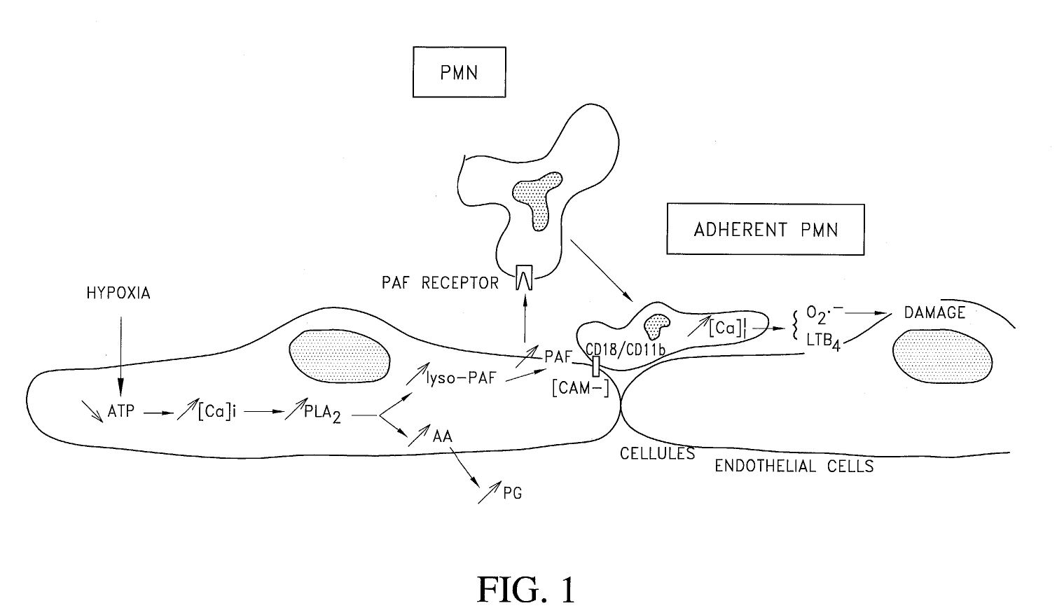

Hypoxia therefore brings about a marked activation of the ECs, which then release prostaglandins and synthesize large quantities of PAF. This activation pathway is summarized in FIG. 1. This synthesis of inflammation mediators can have important repercussions on vascular homeostasis by modulating the functions of the different cell types with which the ECs are in contact.

In order to visualize in more detail what these repercussions might be, a study is made of the adherence of one particular type of leukocyte, i.e. polymorphonuclear neutrophils (PMNs). When ECs are subjected to hypoxia in vitro, their adhesiveness for PMNs is strongly increased. This adherence is in part due to the synthesis of PAF by the hypoxia-activated ECs [Arnould et al., 1993]. While it is not only PMNs which become adherent to hypoxic ECs, it is this adherence which is responsible for activating them [Arnould et al., 1994] (FIG. 1).

It is also known that the ECs synthesize vasoactive molecules which modulate the functions of the SMCs. Experiments suggest that ECs which are activated by a lack of oxygen release various factors which are mitogenic for the SMCs (prostaglandin F₂ₐ and basic fibrosblast growth factor), which in turn induces the cells to proliferate [Michiels et al., 1994].

### Presumed origin of the venous disorder

In light of the results described when using the experimental model in which ECs are exposed to hypoxia in vitro, the origin of the abovementioned pathologies appears to be based on a cascade of events which is initiated by this hypoxia and which ultimately leads to the structural and functional alterations which are seen in the vascular systems.

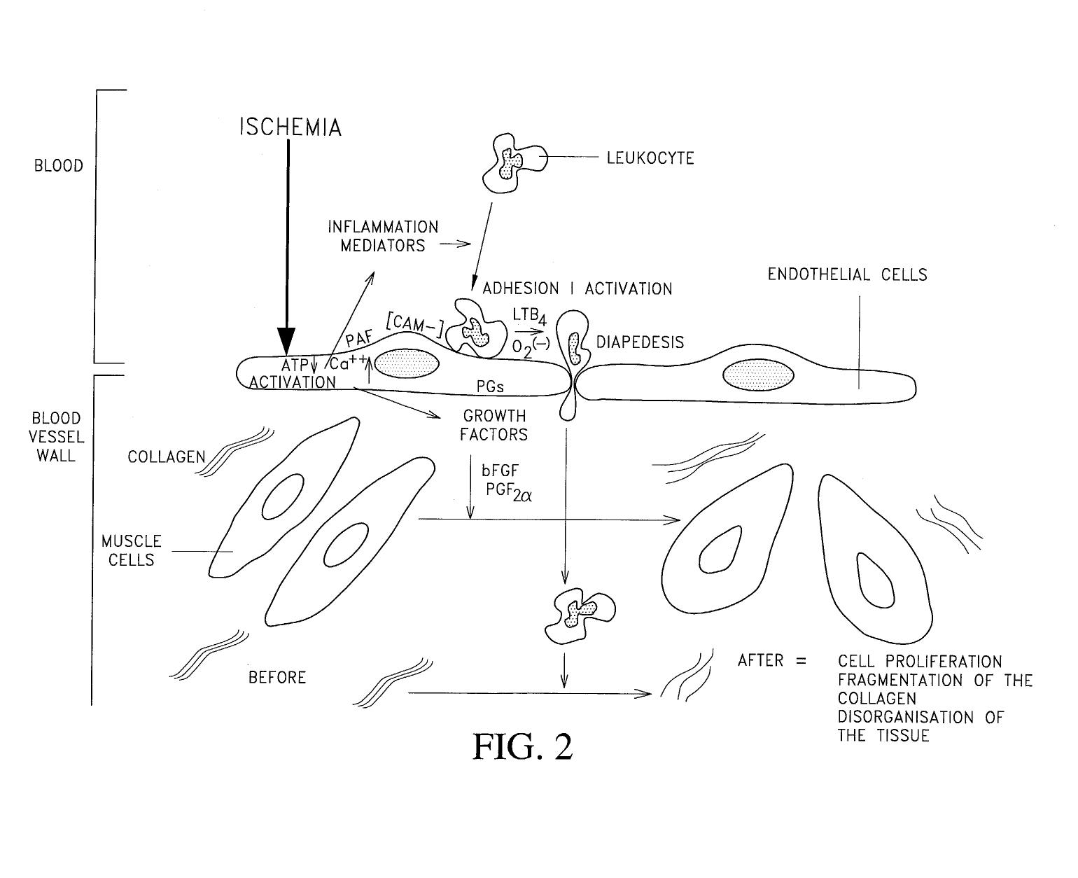

FIG. 2 illustrates this hypothesis. Since it disrupts the blood circulation, venous stasis engenders a decrease in the supply of oxygen and therefore hypoxia. This hypoxia is able to activate the ECs, which form the first layer of the vein wall. These cells then release various inflammatory and mitogenic molecules. The inflammatory molecules are able to induce the adherence of particular leukocytes. This is true not only in the case of the ECs in culture but also in the case of the endothelium of a complete human vein, whether this is an umbilical vein [Arnould et al., 1995] or a saphenous vein. Furthermore, during the adhesion process, the neutrophils are activated and are able to release proteases and free radicals. These molecules are known to have the ability to break down a large number of biological molecules, including components of the extracellular matrix such as collagen.

On the other hand, the hypoxia-activated ECs also synthesize factors which are mitogenic for the SMCs, and which induce the cells to proliferate. Furthermore, it is known that the SMCs which proliferate have a synthetic phenotype, in contrast to the contractile phenotype which is normally present in the wall of normal veins. When they are synthetic, SMCs synthesize more components of the extracellular matrix and lose the ability to express contractile filaments. Proliferation and increased synthesis of the components of the extracellular matrix lead to a thickening of the vein wall, while the loss of actin filaments accounts for the loss of the overall contractility of the vein.

Based on the experimental results obtained from the model of the hypoxia-exposed ECs, a novel hypothesis is proposed with regard to the origin of the abovementioned pathologies: this would be a cascade of cellular interactions which involves leukocytes and SMCs and is initiated by the activation of the ECs by the venous stasis and which would ultimately lead to the structural and functional modifications which are observed.

### Biochemical mechanism of action of the active compounds

Use of the compounds of the invention is seen to result, for example, in an inhibition of the decrease in the content of ATP, in the activation of phospholipase A₂ and in the adhesion of the PMNs which are induced by the hypoxia. The compounds of the invention are also able to inhibit the adhesion of the PMNs to the endothelium of a complete human umbilical vein when the latter is incubated under hypoxic conditions. These results clearly indicate that these compounds have a marked protective action on endothelium which is subjected to hypoxia.

The ability of these medicaments to inhibit the activation cascade of the hypoxia-induced ECs is due to their inhibitory effect on the fall in the content of ATP. This decrease is the event which initiates the activation of the ECs since it is directly coupled to the entry of calcium ions into the cell.

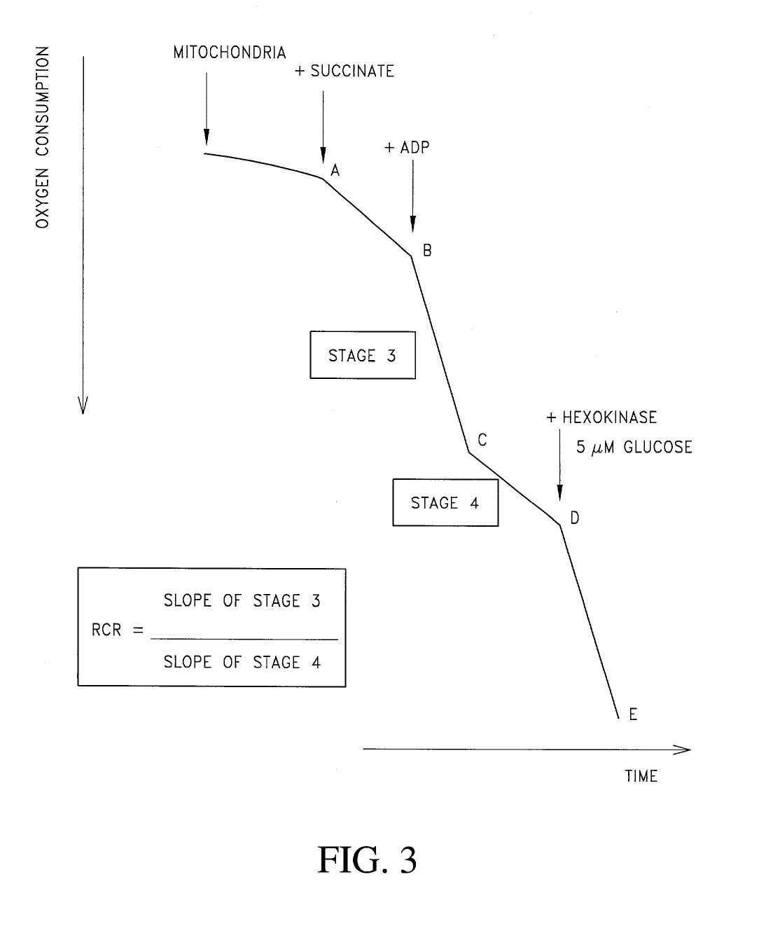

Two hypotheses can be envisaged to explain why these compounds are able to maintain the content of ATP in the ECs under hypoxia: either the compounds activate glycolysis, or they safeguard mitochondrial respiratory activity. The inventors have discovered that these compounds do not activate glycolysis in ECs which are subjected to hypoxia but rather that they would retard the activation of the glycolysis which is directly induced by the hypoxia. These results suggest that the compounds could act on the mitochondria by maintaining a respiratory activity under hypoxia for a longer period of time. This hypothesis is confirmed by measuring the respiratory activity, as expressed by the respiratory control (RCR) (FIG. 3), of liver mitochondria from rats which have been treated per os.

### Effect of bilobalide on brain mitochondria under normal conditions

Different concentrations of active compounds of the invention (bilobalide) were tested on the RCR of the mitochondria which were isolated from rat brains. Five concentrations were employed: 4, 6, 8, 10 and 12 mg/kg of treated patient (rat). The rats were given these doses of bilobalide orally for 14 days. The mitochondria were isolated using the method described by Nowicki et al. (J. Cerebral Blood Flow and Metabolism, 2, pp. 33-40 (1982)). The respiration of the mitochondria is measured in a Clark oxygen electrode which is connected to a recording device. The RCR represents the respiratory control. It denotes the ratio between the consumption of oxygen in the presence of endogenous substrates (glutamate/malate) and the consumption after the ADP has been phosphorylated to ATP. This technique was developed by Chance and Williams (Nature, 175, pp. 1120-1121 (1955)). A dosedependent effect is obtained, with an increase of the RCR, of 3.7 in the case of the controls, to an RCR of 4.6 in the case of the 8 and 10 mg/kg concentrations. A maximum increase of 24% is obtained at 10 mg/kg. These results clearly show that this product has a protective effect on mitochondrial respiration.

### Effect of bilobalide on brain mitochondria in a situation of ischemia

A 15 minute ischemia was performed on control rats and rats which have been treated with active compounds of the invention (bilobalides) for 14 days. The ischemia is performed by decapitation. The rats were treated per os with bilobalide doses of 10 mg/kg for 14 days. When the RCR is measured in the presence of glutamate/malate in the case of a 15 minute ischemia, an RCR of 3 is observed in the case of the controls, with an RCR of 3.9 being observed in the case of the treated rats. The active compound therefore has a protective action on the decrease in respiratory activity which is induced by the ischemia. This protection is manifested through the activity of complex I and of the mitochondrial transport chain. The level of respiratory control which is measured in the presence of glutamate/malate indirectly mirrors the activity of complex I of the electron transfer chain.

The measurement carried out on the mitochondrial complex I was performed by first of all sonicating the mitochondria in order to enable the assay substrates to gain access to the complex I. The latter is assayed by following the reduction of ferricytochrome C at 550 nm. The mitochondrial suspension, which is present in a 25 nM K phosphate buffer, pH 7.4, containing MgCl₂, 10 μm of cytochrome C and 2.5 mg of bovine albumin/ml, is sonicated for 30 seconds at 0° C. 2 mM of KCW [sic] is added and the reaction is started by adding 7.5 mM NADH. The mitochondria are incubated at 37° C. and the reduction of the ferricyanide [sic] is followed at 550 nm. A correction is made for the reduction of the cytochrome C in the presence of rotenone, which inhibits complex I. The results are expressed in μmol of ferricytochrome C reduced per minute.

The activity of complex I was measured on the mitochondria of rats which had been treated per os for 14 days with 10 mg of bilobalide/kg, and after a 15minute ischemia of the brain. An activity of 36 mU/mg of protein is obtained in the case of the control rats, whereas the mitochondria from treated rats have an activity of 44 mU/mg of protein. The activity of the mitochondrial complex I is therefore seen to be significantly protected. A similar protection of complex I is also observed in the case of mitochondria which are isolated from the brains of rats which have not undergone a period of ischemia.

### Effect of bilobalide on liver mitochondria

The active compound of the invention (bilobalide) was administered per os to rats for 14 days at concentrations of 8 mg/kg of patient (rat). The liver mitochondria of these rats were isolated using the method described by Remacle (J. Cell. Biol. 79, 291, 1978). The respiratory activity of the mitochondria of treated rats exhibited an RCR of 13.25, as compared with an RCR of 7.6 in the case of the control rats. A 10 minute ischemia was performed on the livers by perfusion in a medium consisting of 0.137 M NaCl, 5.4 mM KCl, 0.8 mM MgSO₄, 11 mM glucose, 0.34 mM Na₂HP0₃, 24.4 mM NaHCO₃, 6.35 mM KH₂PO₄ and 8 mg of bilobalide/l. The medium is first of all degassed under an atmosphere containing 95% N₂ and 5% CO₂. The mitochondria are isolated and their respiratory activity is determined.

For the controls, the rats were given water for 14 days and the livers were perfused with the same solution as for the tests. An RCR of 5.24 was observed in the case of the treated rat mitochondria and of 3.73 in the case of the control rats.

These results demonstrate that the active compound of the invention (bilobalide) possesses an anti-ischemic activity which is manifested both in vitro (ECs which are subjected to hypoxia) and in vivo (hepatic and cerebral ischemia in treated rats), and that this activity is at least partly due to protection of the mitochondria, which increase their respiratory activity and therefore their synthesis of ATP.

Each of the active compounds of the invention can, like bilobalide, increase the RCR of mitochondria which have been isolated from the livers of untreated rats when the mitochondria are preincubated for one hour in the presence of these medicaments. The active compounds can therefore act directly on the mitochondria. It is very interesting to note that it is possible to separate the medicaments, on the basis of these results, into two different classes: melilot extract, Ruscus extract, procyanidine oligomers and hydroxyethylrutosides, which increase the RCR by increasing stage 3 of respiration (class II), whereas bilobalide, aescin, nafthoquinone and diosmin, which constitute the medicaments of class I, increase the RCR by decreasing stage 4 of respiration (see table 3).

TABLE 3
Effect of the medicaments on the protection of
the mitochondria

| | | | Protective | |
|---|---|---|---|---|
| Class I | Final | | effect on | Increase of |
| molecules | concentrations | | the RCR (%) | stage 3 (%) |
| Ginkor Fort | 0.3 | mg/ml | +28.1 | −21 |
| Bilobalide | 0.8 | μg/ml | +22.7 | −41 |
| Reparil | 75 | ng/ml | +18.1 | −43 |
| Naphthoquin | 10⁻⁷ | M | +19.7 | −44 |
| one | | | | |
| Praxilene | 10⁻⁷ | M | +17.7 | −35 |

| | | Protective | |
|---|---|---|---|
| Class II | Final | effect on | Decrease of |
| molecules | concentrations | the RCR (%) | stage 4 (%) |
| Venoruton | 0.1 mg/ml | +23.3 | +41 |
| Cyclo 3 | 4.5 μg/ml | +14.5 | +20 |
| Endotelon | 1.5 μg/ml | +15.3 | +82 |
| Esberiven | 1 mg/ml | +15.5 | +12 |

Different sites of action therefore appear to be involved and lead to an increase in mitochondrial respiratory activity and thus to a decrease [sic] in the content of ATP during hypoxia.

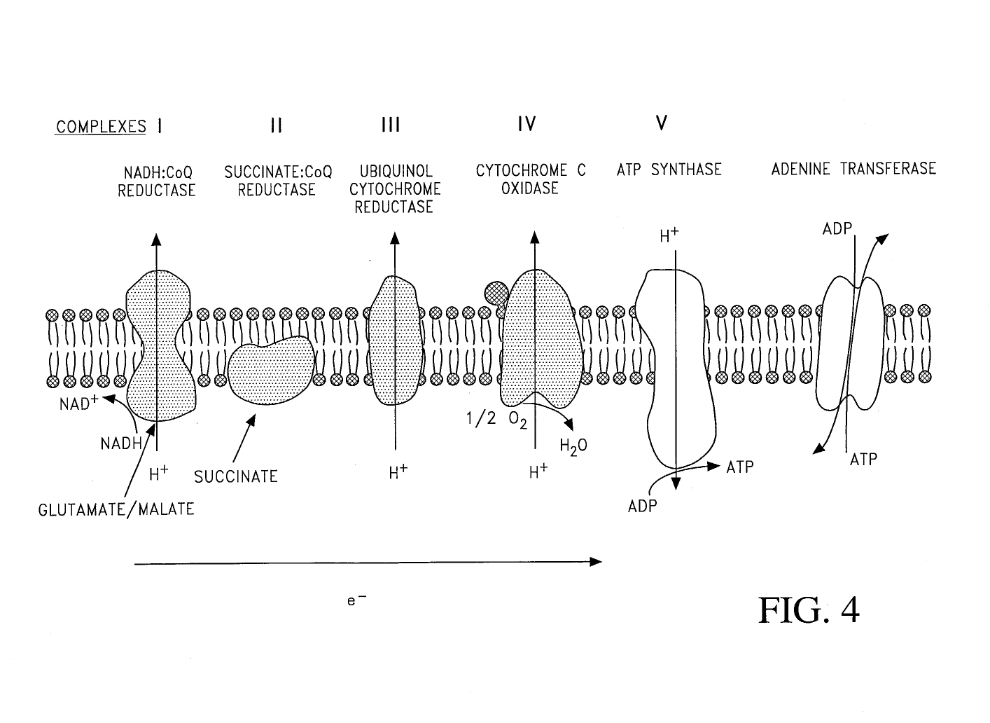

The process of the oxidative phosphorylation of ATP is a complex process which involves different enzyme complexes or electron transporters, which generate a proton gradient (complexes I, III and IV), ATP synthases (complex V), which is directly responsible for synthesizing ATP, and adenine translocase, which is the transporter which is required for importing ADP and exporting ATP (FIG. 4). In order to demonstrate what -the enzyme target of these medicaments might be, each complex was inhibited by a specific inhibitor and a study was carried out, while measuring the RCR, to determine whether these preparations were able to relieve this inhibition. The results show that none of these medicaments has any effect on complexes IV and V. On the other hand, the class I medicaments, which increase the RCR by decreasing stage 4, strongly protect complex III and, to a lesser degree, complex I (see Table 4).

TABLE 4
Protective effect of the class I medicaments
on complexes I and III of the mitochondria

| | | | Protection | Protection |
|---|---|---|---|---|
| Class I | Final | | of complex | of complex |
| molecules | concentrations | | I (%) | III (%) |
| Ginkor Fort | 0.3 | mg/ml | 37.7 | 38.1 |
| Bilobalide | 0.8 | μg/ml | 27.5 | 47.9 |
| Reparil | 75 | ng/ml | 19.6 | 33.2 |
| Nafthoquinone | 10⁻⁷ | M | 14.1 | 46.6 |
| Praxilene | 10⁻⁷ | M | 33.5 | 43.6 |

Complex I of the mitochondria was 58% inhibited by the presence of 0.5 mM Amytal. Under these conditions, the presence of the molecules at the indicated concentrations (60 minute incubation) results in an inhibition which is markedly less pronounced, with this having been expressed in percentage protection as compared with the mitochondria without medicament, which are regarded as having 0% protection.

Furthermore, it was demonstrated, by directly measuring the activity of adenine translocase, that the activity of this transporter is increased by the class II medicaments which increase stage 3 of respiration (see table 5).

TABLE 5
Effect of the class II medicaments on the
adenine translocase activity of the mitochondria

| | | Increase in |
|---|---|---|
| | Final | adenine |
| Molecules | concentrations | translocase |
| Endotelon | 3 μg/ml | 102.4 ± 5.2 |
| Venoruton | 0.1 mg/ml | 110.4 ± 15 |

The effect of the medicaments on the activity of the mitochondrial adenine translocase was determined by measuring the transport of (c¹⁴) ADP into the mitochondria over 45 seconds.

Furthermore, while the protective action of cyclo 3 on the mitochondrial RCR is linked to the increase in the mitochondrial RCR, it is without doubt due to its protective effect on mitochondrial uncoupling. Thus, in the presence of cyclo 3, the mitochondria are seen to have a better resistance to uncoupling with 0.5 and 1 μM mCCP with this protective efect being of the order of 20%.

Another interesting property of the compounds of the invention is that it is possible to achieve a synergistic effect by combining different molecules which act on different targets, for example a molecule which acts on complexes I and III, with this action finding expression in a decrease in stage 4 of mitochondrial respiration, and one or more molecules which act on adenine translocase, with this action finding expression in an increase in stage 3 of mitochondrial respiration. The double protection which is thus obtained yields an overall result which is advantageous and unexpected since it exceeds the maximum protection which is possible with each of the compounds.

Two enzyme targets of the active compounds have been identified. The consequence of the action of the compounds on these two targets is to increase the production of ATP by the mitochondria and to prevent this production from decreasing under ischemic conditions. Thus, the active compounds protect the cells from the consequences of an energy deficit, which deficit can, in the case of the ECs, lead to their activation, and to the recruitment, adhesion and activation of leukocytes and to proliferation of the SMCs (FIG. 2).

These molecules all possess the property of maintaining a high level of ATP production by the mitochondria, even under unphysiological situations such as periods of ischemia or of a decrease in these mitochondrial activities due to age or to pathologies associated with ageing, thereby enabling the molecules to be applied in these various pathologies.

What is claimed is:

1. A method for therapeutically and/or prophylactically treating ischemia and/or the pathologies associated with ischemia or an energy deficit in a patient comprising administering to said patient a pharmaceutical composition comprising bilobalide and a pharmaceutically acceptable excipient whereby said composition has a therapeutic and/or prophylactic effect on ischemia and/or the pathologies associated with ischemia or an energy deficit.

2. The method of claim 1 wherein said bilobalide is comprised in a Ginkor fort composition.

3. The method of claim 1 wherein said pathologies associated with ischemia are selected from the group consisting of myocardial infarction, cerebral ischemia, chronic venous insufficiency, atheriopathies, Raynaud's phenomenon, loss of auditory acuity, disorders associated with time spent at high altitude, ulcers, change in capillary permeability, capillary fragility, wound-healing, changes to the skin, angina pectoris engendered by short periods of coronary obstruction, pulmonary hypertension, hepatic ischemia, Parkinson's disease, diabetes and heart transplants.

4. The method of claim 2 wherein said pathologies associated with ischemia are selected from the group consisting of myocardial infarction, cerebral ischemia, chronic venous insufficiency, atheriopathies, Raynaud's phenomenon, loss of auditory acuity, disorders associated with time spent at high altitude, ulcers, change in capillary permeability, capillary fragility, wound-healing, changes to the skin, angina pectoris engendered by short periods of coronary obstruction, pulmonary hypertension, hepatic ischemia, Parkinson's disease, diabetes and heart transplants.
